(19) United States
(12) Patent Application Publication
(10) Pub. No.: US 2005/0154229 A1
(43) Pub. Date: Jul. 14, 2005

(54) Maleic acid salt of(2s)-n-{5-[amino(imino)methy]-2-thienyl}methyl-1-{(2r)-2-[(carboxymethyl)amino]-3,3-diphenylpropanoyl}-2-pyrrolidine carboxamide and a process for

(76) Inventors: Aeri Kim, Taejeon (KR); Jae-Hyeon Park, Taejeon (KR); Suk-Kyoon Yoon, Taejeon (KR); Cheol-Won Park, Taejeon (KR); Kwan-Hyung Cho, Taejeon (KR)

Correspondence Address: BIRCH STEWART KOLASCH & BIRCH, PO BOX 747, FALLS CHURCH, VA, 22040-0747, US

(21) Appl. No.: 10/508,485

(22) PCT Filed: Mar. 21, 2003
(86) PCT No.: PCT/KR03/00559

(30) Foreign Application Priority Data
Mar. 22, 2002 (KR) 10-2002-0015661

(51) Int. Cl. C07C 57/02

(52) U.S. Cl. 562/595

(57) ABSTRACT

The present invention relates to a maleic acid salt of (2S)-N-{5-[amino(imino)methyl]-2-thienyl}methyl-1-{(2R)-2-[(carboxymethyl)amino]-3,3-diphenylpropanoyl}-2-pyrrolidinecarboxamide.

### TECHNICAL FIELD

The present invention relates to a maleic acid salt of (2S)-N-{5-[amino(imino)methyl]-2-thienyl}methyl-1-{(2R)-2-[(carboxymethyl)amino]-3,3-diphenylpropanoyl}-2-pyrrolidine carboxamide represented by the following Formula (1), and a process for preparing the same:

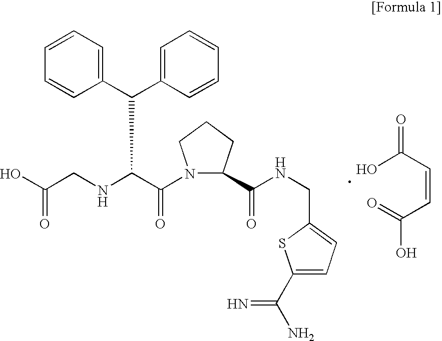

### BACKGROUND OF THE INVENTION

The free compound of Formula (1), i.e., compound to which acids were not added, and pharmaceutically acceptable salts, hydrates, solvates, and isomers thereof are the subjects of Korean Patent Laid-Open Publication No. 2000-047461 and WO 0039124, and may be effectively used as new thrombin inhibitors.

If a drug does not have physical properties suitable for its development, several methods may be used to improve them. Especially, in case that a drug's solubility is low, preparation of salts of the drug is used as a general method to improve the solubility. Methods to prepare its salts have been conventionally well known (for instance, Pharmaceutical Salts, Journal of Pharmaceutical Sciences, Donald C. Monkhouse et al, 1, 66(1), 1977; and Salt selection for basic drugs, International Journal of Pharmaceutics, Philip L. Gould, 201, 33, 1986).

The physical property of a drug has a huge effect on production and development process of its raw drug and development process of its final product. A drug in the form of solid may have several advantages, such as easy to handle and store, and easy to control its quality. Furthermore, in the development process of the drug's final product, designing dosage forms and administration forms thereof may be easy. A drug in solid may be roughly divided by crystalline form and amorphous form according to its crystallinity. Some drugs may be obtained in both crystalline form and amorphous form, while other drugs may be obtained only in either crystalline form or amorphous form. Crystalline form and amorphous form may exhibit large difference in physicochemical properties. For instance, it has been already reported that some drugs show a difference in solubility and bioavailability depending on crystalline form or amorphous form (for example, Pharmaceutical Solids: A Strategic Approach to Regulatory Considerations, Stephen Byrn et al, Pharmaceutical Research, 945, 12(7), 1995). Therefore, for the reasons explained above, crystallinity of a drug is very important for its preparation and administration.

Except special cases, it is easy to obtain a drug having crystallinity in the process of research and development. A report already shows that crystallinity of a drug may be an important advantage in that in the final step to produce the drug, the drug may be purely obtained through recrystallization that is a relatively easy purification process, and a crystalline drug, whose physicochemical properties may be easily identified, is advantageous even in the quality control of its product process (see, An integrated approach to the selection of optimal salt form for a new drug candidate, Abu T. M. Serajuddin et al, International Journal of Pharmaceutics, 209, 105, 1994). Thus, in case that a drug is obtained in the amorphous form, to crystallize the drug is very important for its development and production. Also, since deliquescence of a drug makes its production and quality control difficult, it is preferable that a drug is not deliquesced. The same is applicable to a drug's salts, and so salts having suitable solubility, and crystallinity, not deliquescence, among synthesized various salts are preferred.

Some compounds may have difficulty in being absorbed due to a low dissolution rate. To confirm the above difficulty beforehand, a disk having the area of 0.5 cm² is prepared, and then its dissolution rate in various media is measured. The dissolution amount over time is measured and its value is divided by the disk's area. The value is the dissolution rate per unit area. If the value is usually 1 mg/min/cm² or more, it can be said that there is no phenomenon for the drug not to be absorbed due to the low dissolution rate. If the value is 0.1 mg/min/cm² or less, it can be said that the drug has a problem of absorption due to the low dissolution rate (see, Howard C. Ansel, Nicholas G. Popovich and Loyd V. Allen, 1995, Pharmaceutical Dosage Forms and Drug Delivery Systems, 6th ed., Williams & Wilkins, pp.109 and Jens T. Carstensen, 1996, Modern Pharmaceutics: Gilbert S. Banker and Christopher T. Rhodes (Ed.), Drugs and the pharmaceutical sciences, Vol. 72, 3rd ed., Dekker, pp. 233.). Thus, measuring the dissolution rate per unit area has a very significant meaning in predicting the absorption problem that may occur in in vivo test of the drug later. Therefore, a compound having an excellent dissolution rate per unit area should be selected as a candidate for the drug development.

### BRIEF SUMMARY OF THE INVENTION

The inventors have carefully and extensively studied to find out a drug's salts having crystallinity and the most excellent physical properties, such as superior solubility, no deliquescence, and a superior dissolution rate per unit area, among salts synthesized so far. Finally, they found out that the maleic acid salt of a drug is the most suitable, and complete the present invention.

Accordingly, the object of the present invention is to provide maleic acid salt of (2S)-N-{5-[amino(imino)methyl]-2-thienyl}methyl-1-{(2R)-2-[(carboxymethyl)amino]-3,3-diphenylpropanoyl}-2-pyrrolidine carboxamide represented by the following Formula (1):

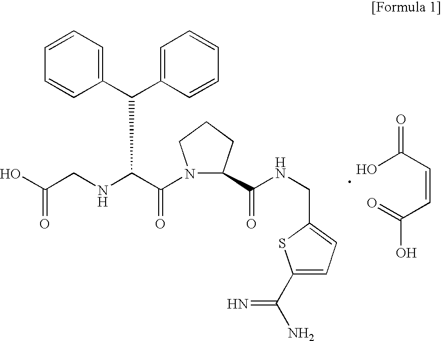

Another object of the invention is to provide a process for preparing the compound of Formula 1, characterized in that the free compound of Formula 1 is reacted with maleic acid in the presence of alcohol solvent(s).

A further object of the invention is to provide a process for preparing the crystalline form of the compound of Formula 1, characterized in that the free compound of Formula 1 is reacted with maleic acid in the presence of alcohol solvent(s) to obtain the amorphous form of maleic acid salt of Formula 1, and then the maleic acid salt is recrystallized.

### BRIEF DESCRIPTION OF DRAWINGS

FIG. 1 is a powder X-ray diffraction diagram of crystalline form of maleic acid salt of (2S)-N-{5-[amino(imino)methyl]-2-thienyl}methyl-1-{(2R)-2-[(carboxymethyl)amino]-3,3-diphenylpropanoyl}-2-pyrrolidine carboxamide.

### DETAILED DESCRIPTION

The Detailed Description of the present invention is provided below.

The Formula 1 according to the present invention may be prepared by reacting the free compound of Formula 1 with maleic acid in the presence of alcohol solvent(s). The free compound of Formula 1 used in the reaction is prepared according to a method described in Korean Patent Laid-Open Publication No. 2000-047461 and WO0039124. The solvent(s) to be used in the reaction may be usually available kinds of alcohols, which are alcohols of alkanes having the carbon number of 1 to 8, such as methanol, ethanol, propanol, butanol, isopropanol, octanol, etc., preferably methanol and ethanol, and most preferably methanol, but not limited to them.

The maleic acid salt according to the present invention is obtained in the amorphous form through the above reaction, but may be also obtained in the crystalline form through recrystallization by use of suitable solvent(s). Solvents to be used in preparation of the amorphous form or recrystallization may be usually available kinds of alcohols, which are alcohols of alkanes having the carbon number of 1 to 8, such as methanol, ethanol, propanol, butanol, isopropanol, octanol, etc., preferably methanol and ethanol, and most preferably methanol, but not limited to them. Furthermore, the solvents to be used for recrystallization may be, in addition to alcohols exemplified above, water and organic solvents, for example, n-hexane, ethylacetate, butylacetate, acetonitril, chloroform, diethylether, acetone, etc., and other usually available solvents. The above free compound may be dissolved or dissolved in heating, by using one solvent or more than one in mixture among the above, and may be recrystallized. It has been found that the above maleic acid salt is not deliquesced even at any relative humidity, a weight change of the salt is not relatively large, and the dissolution rate of the salt is more remarkably excellent than any of the free compound since the maleic acid salt has been crystallized. This conforms that the maleic acid salt is superior to the free compound with respect to solubility, dissolution, and absorption in a gastrointestinal tract.

Because the free compound may be effectively used as a thrombin inhibitor as described in Korean Patent Laid-Open Publication No. 2000-047461 and WO 0039124, the maleic acid salt according to the present invention is also useful as a thrombin inhibitor.

Below, the present invention will be explained with the following examples, comparative examples, and test examples in more detail. However, it should be understood that these examples have been described as preferred specific embodiments of the present invention, and are not intended to limit the scope of the present invention in any way. Other aspects of this invention will be apparent to those skilled in the art to which the present invention pertains.

### EXAMPLES

### Example 1

Preparation of amorphous form of maleic acid salt of (2S)-N-{5-[amino(imino)methyl]-2-thienyl}methyl-1-{(2R)-2-[(carboxymethyl)amino]-3,3-diphenylpropanoyl}-2-pyrrolidine carboxamide

The free compound of Formula 1 (1 g) was dissolved in methanol (30 ml) and then water (30 ml) was added thereto. An equivalent of maleic acid was added thereto dropwise and then the mixture was stirred for one hour. Solvent therein was removed by a distillator under reduced pressure to obtain the-titled amorphous form of maleic acid salt (1.1 g, yield 95%).

### Example 2

Preparation of the crystalline form of maleic acid salt of (2S)-N-{5-[amino(imino)methyl]-2-thienyl}methyl-1-{(2R)-2-[(carboxymethyl)amino]-3,3-diphenylpropanoyl}-2-pyrrolidine carboxamide (1)

The free compound of Formula 1 (3 g) was dispersed in methanol (50 ml), 1 M solution of maleic acid in methanol (30 ml) was added thereto and then the mixture was stirred for 0.5 hour. Thereafter, acetonitril (300 ml) was added thereto. The mixture was stirred for one hour and then placed to obtain white crystals. The crystals were filtered, washed by acetonitril and then dried under vacuum (2.25 g, yield 61.7%).

### Example 3

Preparation of the crystalline form of maleic acid salt of (2S)-N-{5-[amino(imino)methyl]-2-thienyl}methyl-1-{(2R)-2-[(carboxymethyl)amino]-3,3-diphenylpropanoyl}-2-pyrrolidine carboxamide (2)

The free compound of Formula 1 (2.3 g) was dispersed in ethanol (100 ml), 1 M solution of maleic acid in methanol (4.3 ml) was added thereto and then the mixture was stirred for 0.5 hour. Thereafter, acetonitril (500 ml) was added thereto. The mixture was stirred for one hour and then placed to obtain white crystals. The crystals were filtered, washed by acetonitril and thereafter dried under vacuum (1.04 g, yield 38.3%).

### Example 4

Preparation of the crystalline form of maleic acid salt of (2S)-N-{5-[amino(imino)methyl]-2-thienyl}methyl-1-{(2R)-2-[(carboxymethyl)amino]-3,3-diphenylpropanoyl}-2-pyrrolidine carboxamide (3)

The free compound of Formula 1 (3.9 g) was dissolved in isopropanol (100 ml), 1 M solution of maleic acid in methanol (7.2 ml) was added thereto and then the solution was stirred for 0.5 hour. Thereafter, acetonitril (500 ml) was added thereto. The mixture was stirred for one hour and then was placed to obtain white crystals. The crystals were filtered, washed by acetonitril and then dried under vacuum (3.3 g, yield 72.8%).

### Example 5

Recrystallization of the amorphous form of maleic acid salt of (2S)-N-{5-[amino(imino)methyl]-2-thienyl}methyl-1-{(2R)-2-[(carboxymethyl)amino]-3,3-diphenylpropanoyl}-2-pyrrolidine carboxamide

The amorphous form of maleic acid salt prepared in Example 1 (1 g) was dissolved in methanol (20 ml). Thereafter, acetonitril (80 ml) was added thereto and the solution was placed to obtain white crystals. After filtering, they were washed by acetonitril and dried under vacuum (0.62 g, yield 62%).

### Test Example 1

Powder X-ray diffraction test on the crystalline form of maleic acid salt of (2S)-N-{5-[amino(imino)methyl]-2-thienyl}methyl-1-{(2R)-2-[(carboxymethyl)amino]-3,3-diphenylpropanoyl}-2-pyrrolidine carboxamide

40 mg of the crystalline form of maleic acid salt prepared in Example 2 was thinly coated onto a sample holder and then the powder X-ray diffraction test was conducted according to the following conditions. By using Rigaku Geigeflex D/max-III C apparatus, the test was conducted at 35 kV, 20 mA.

Scan speed (2Θ)) 5/min

Sampling time 0.03 sec

Scan mode continuous

Cu-target (Ni filter)

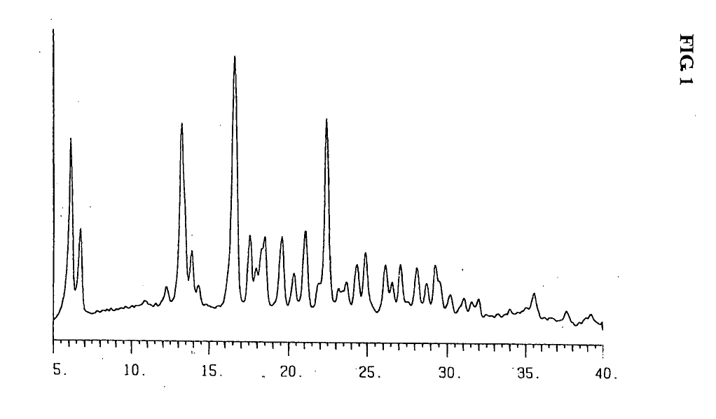

The result of the powder X-ray diffraction test is shown in FIG. 1 and the positions of peaks shown in the above figure are listed at Table 1.

TABLE 1
Peaks of the powder X-ray diffraction of maleic acid salt of
(2S)-N-{5-[amino(imino)methyl]-2-thienyl}methyl-1-
{(2R)-2-[(carboxymethyl)amino]-3,3-diphenylpropanoyl}-
2-pyrrolidine carboxamide
peak
2⊖

6.149
6.754
12.254
13.261
13.91
14.345
16.661
17.577
17.966
18.506
19.592
20.368
21.082
22.445
23.198
23.701
24.364
24.897
26.171
26.601
27.127
28.16
28.76
29.292
30.212
31.077
31.559
32
34.005

### Test Example 2

Moisture absorption and dehumidification tests of the amorphous form of maleic acid salt of (2S)-N-{5-[amino(imino)methyl]-2-thienyl}methyl-1-{(2R)-2-[(carboxymethyl)amino]-3,3-diphenylpropanoyl}-2-pyrrolidinecarboxamide

After 40 mg of the amorphous form of maleic acid salt prepared in Example 1 was thinly coated onto a beaker, the sample was moisture-absorbed by placing it at each relative humidity of 33%, 57%, 64%, 75% and 93% for two days or more and its condition was observed. In order to obtain each relative humidity above, as shown in the table below, saturated aqueous solutions of salts were prepared and then placed in a desiccator, and the desiccator was sealed.

TABLE 2

| | |
|---|---|
| Relative Humidity 33% | MgCl₂ saturated aqueous solution |
| Relative Humidity 57% | Mg(NO₃)₂.6H₂O saturated aqueous solution |
| Relative Humidity 64% | NaNO₂ saturated aqueous solution |
| Relative Humidity 75% | NaCl saturated aqueous solution |
| Relative Humidity 93% | KNO₃ saturated aqueous solution |

The amorphous form of maleic acid salt of the free compound was hardened at the relative humidity of 75% and was deliquesced at the relative humidity of 93%.

### Test Example 3

Moisture absorption and dehumidification tests of the crystalline form of maleic acid salt of (2S)-N-{5-[amino(imino)methyl]-2-thienyl}methyl-1-{(2R)-2-[(carboxymethyl)amino]-3,3-diphenylpropanoyl}-2-pyrrolidinecarboxamide

100 mg of the crystalline form of maleic acid salt prepared in Example 2 was thinly coated onto a beaker and then placed at each relative humidity of 33%, 57%, 64%, 75% and 93% for two days or more to be moisture-absorbed. Thereafter, the weight change of the sample was measured while its condition is observed. In order to obtain each relative humidity above, as shown in Table 2 of Test example 2, saturated aqueous solutions of salts were prepared and then placed in a desiccator and the desiccator was sealed.

The weights on moisture absorption were increased by 1.4%, 3.0%, 4.3%, 4.1%, and 6.7% based on the form's initial weight, at the relative humidity of 33%, 57%, 64%, 75%, and 93%, respectively, and also on dehumidification the weights were similarly changed to the moisture absorption. Differently from the amorphous form of maleic acid salt, the crystalline form of maleic acid salt was not deliquesced even at the relative humidity of 93%.

### Test Example 4

Powder X-ray diffraction test during moisture absorption and dehumidification of the crystalline form of maleic acid salt of (2S)-N- {5-[amino(imino)methyl]-2-thienyl}methyl-1-{(2R)-2-[(carboxymethyl)amino]-3,3-diphenylpropanoyl}-2-pyrrolidine carboxamide

40 mg of the maleic acid salt prepared in Example 2 was thinly coated onto a sample holder. Immediately after the sample was vacuum dried in the presence of P₂O₅, and after the sample was placed for moisture absorption at each relative humidity of 33%, 53%, 64%, 75% and 93% for two days or more, respectively, the powder X-ray diffraction test of the sample was conducted according to the conditions represented in the above Test example 1 to observe change of the crystalline form during moisture absorption. While lowering the relative humidity, the same test was repeated to observe change of the crystalline form during dehumidification. In order to obtain each relative humidity above, as shown in Table 2 of Test example 2, saturated aqueous solutions of salts were prepared and then placed in a desiccator and the desiccator was sealed. The powder X-ray diffraction diagram of the crystalline form of maleic acid salt did not show any change during moisture absorption and dehumidification.

### Test Example 5

Test of the dissolution rate per unit area of the crystalline form of maleic acid salt of (2S)-N-{5-[amino(imino)methyl]-2-thienyl}methyl-1-{(2R)-2-[(carboxymethyl)amino]-3,3-diphenylpropanoyl}-2-pyrrolidinecarboxamide

200 mg of the crystalline form of maleic acid salt prepared in Example 2 was put into a mold. By applying pressure on the mold, a disk having the area of 0.5 cm² was prepared and the disk was added to each of the buffer solutions (600 ml) of pH 3.8 and 7.4 to conduct the dissolution test. A portion of the solution was periodically taken and its concentration was measured to determine the dissolution rate per unit area. The dissolution rate per unit area of the maleic acid salt was 11.8 mg/min/cm² at pH 3.8 and 1.7 mg/min/cm² at pH 7.4.

### Comparative Example 1

Preparation of diHCl salt

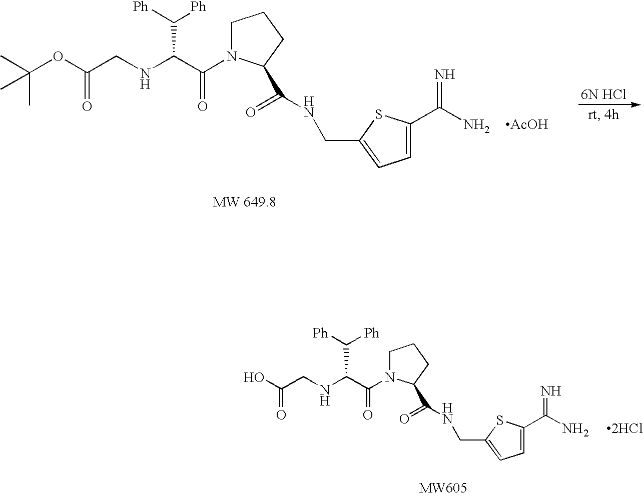

The above starting material (3 g) was added to 6N HCl (90 ml) and then the mixture was stirred for 4 hours (checking completion of the reaction by HPLC). After the solution was concentrated under reduced pressure, 2-propanol (30 ml) was added thereto and the mixture was stirred for 10 minutes while heating until a clear solution was obtained therefrom. The solution was cooled to room temperature and thereafter 30 ml of hexane was slowly added while stirring. White products obtained therefrom were filtered, washed by hexane, and then dried under nitrogen. diHCl salt obtained therefrom was in the amorphous form, and recrystallizing it was tried, but crystalline diHCl salt was not obtained.

### Comparative Example 2

Test of the dissolution rate per unit area of the free compound of (2S)-N-{5-[amino(imino)methyl]-2-thienyl}methyl-1-{(2R)-2-[(carboxymethyl)amino]-3,3-diphenylpropanoyl}-2-pyrrolidine carboxamide

The same test as Test example 5 was conducted on the free compound of Formula 1. The dissolution rate per unit area of the free compound was 1.3 mg/min/cm² at pH 3.8 and 0.3 mg/min/cm² at pH 7.4.

### Comparative Test Example 1

The method as described in Test example 2 was applied to the amorphous diHCl salt prepared in Comparative Example 1. From observing its change at each relative humidity, it is shown that the salt was deliquesced at the relative humidity of 75%. This contrasts with the result of Test example 3 that the maleic acid salt according to the invention was not deliquesced even at the relative humidity of 93%. Therefore, it can be shown that the maleic acid salt is the most excellent among various acid salts.

### Industrial Applicability

Comparing the values from Test Example 5 and Comparative Example 2, the maleic acid salt showed a more remarkably excellent dissolution rate per unit area than the free compound even at neutral pH as well as at pH 3.8. In particular, as confirmed in Test Examples 2, 3 and 4, the crystalline form of maleic acid salt according to the invention has excellent characteristics such as a little change during moisture absorption and dehumidification and no deliquescence at the relative humidity of 93%. Therefore, the maleic acid salt is particularly useful as a thrombin inhibitor.

1. A maleic acid salt of (2S)-N-{5-[amino(imino)methyl]-2-thienyl}methyl-1-{(2R)-2-[(carboxymethyl)amino]-3,3-diphenylpropanoyl}-2-pyrrolidine carboxamide represented by the following Formula 1:

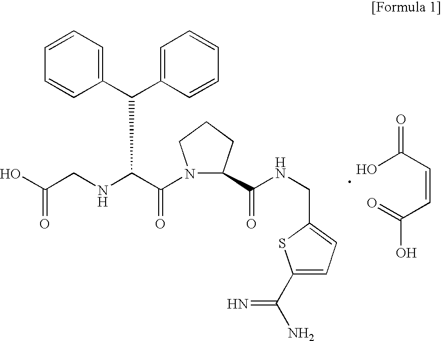

2. A process for preparing the compound of Formula 1 of claim 1, characterized in that the free compound of Formula 1 of claim 1 is reacted with maleic acid in the presence of alcohol solvent(s).

3. A process for preparing the crystalline form of compound of Formula 1 of claim 1, characterized in that the free compound of Formula 1 of claim 1 is reacted with maleic acid in the presence of alcohol solvent(s) to obtain the amorphous form of maleic acid salt of Formula 1, and then the maleic acid salt is recrystallized.

4. The process according to claim 2 or claim 3 wherein the alcohol solvent(s) is one or more selected from a group consisted of methanol, ethanol, propanol, butanol, isopropanol, and octanol.
